(19) United States
(12) Patent Application Publication
(10) Pub. No.: US 2010/0030103 A1
(43) Pub. Date: Feb. 4, 2010

(54) Measuring device for physiological parameters

(75) Inventors: Theodor Lutze, Balgheim (DE); Dirk Schauer, Berlin (DE); Christoph Miethke, Potsdam (DE)

Correspondence Address: Lipsitz & McAllister, LLC, 755 MAIN STREET, MONROE, CT, 06468, US

(73) Assignee: Aesculap AG, Tuttlingen (DE)

(21) Appl. No.: 12/460,447

(22) Filed: Jul. 17, 2009

Related U.S. Application Data
(63) Continuation of application No. PCT/EP2008/000039, filed on Jan. 5, 2008.

(30) Foreign Application Priority Data
Feb. 22, 2007 (DE) 10 2007 008 642

(51) Int. Cl. A61B 5/00

(52) U.S. Cl. 600/561

(57) ABSTRACT

In a measuring device for physiological parameters with an implantable fluid chamber accommodating brain fluid, with a sensor device communicating with the brain fluid in the fluid chamber via a membrane and comprising a sensor for one or more physiological parameters, electronic components and a telemetry device for wireless transmission of signals of the sensor, in order to improve the manufacture and handling, it is proposed that the fluid chamber comprise at least two housing parts which are adapted to be fitted together in a sealed manner, thereby forming a closed interior, and which allow access to the interior when not fitted together, and that the sensor device be arranged in a measurement chamber closed on all sides, which is configured as a component that is adapted for independent handling and is insertable in a defined position in the interior of the fluid chamber.

This application is a continuation of international application number PCT/EP2008/000039 filed on Jan. 5, 2008.

The present disclosure relates to the subject matter disclosed in international application number PCT/EP2008/000039 of Jan. 5, 2008 and German application number 10 2007 008 642.5 of Feb. 22, 2007, which are incorporated herein by reference in their entirety and for all purposes.

### BACKGROUND OF THE INVENTION

The invention relates to a measuring device for physiological parameters with an implantable fluid chamber accommodating brain fluid, with a sensor device communicating with the brain fluid in the fluid chamber via a membrane and comprising a sensor for one or more physiological parameters, electronic components and a telemetry device for wireless transmission of signals of the sensor.

Such a measuring device is described, for example, in WO 2006/117123 A1. Such measuring devices are usually implanted outside the cranial bone and underneath the scalp, in some cases directly over a drill hole in the cranial vault, in other cases next to such a drill hole through which brain fluid can enter the fluid chamber from the interior of the cranium. In the previously known measuring device, the fluid chamber is configured as a double chamber which is divided by an intermediate wall into two compartments, one of the compartments accommodating the sensor device, and the brain fluid flowing through the other compartment. Arranged in the intermediate wall between the compartments is a membrane through which the pressure of the brain fluid is transferred to a pressure senor of the sensor device.

Such an assembly must be prefabricated in its entirety and must be hermetically sealed. Therefore, in the event of a failure, only a complete exchange is possible. Moreover, the manufacture is complex.

The object of the invention is to so construct a generic measuring device that the manufacture is simplified and that, if necessary, repair work can be carried out without having to exchange the entire measuring device.

### SUMMARY OF THE INVENTION

This object is accomplished, in accordance with the invention, in a measuring device of the kind described at the outset in that the fluid chamber comprises at least two housing parts which are adapted to be fitted together in a sealed manner, thereby forming a closed interior, and which allow access to the interior when not fitted together, and in that the sensor device is arranged in a measurement chamber closed on all sides, which is configured as a component that is adapted for independent handling and is insertable in a defined position in the interior of the fluid chamber.

Owing to the construction of the measurement chamber as a component that can be handled independently, it can be inserted into the open interior of the fluid chamber and is then held in a defined position in it. By joining the at least two housing parts of the fluid chamber, the fluid chamber can then be sealed tightly towards the outside, but it can also be opened again, if necessary, so that the measurement chamber can be taken out and, if necessary, exchanged, without exchange of the fluid chamber being required. The fluid chamber may even remain in its implanted position in the event of a failure of the sensor device and can continue to be used after insertion of a new measurement chamber.

Therefore, the surgeon also has the possibility of not inserting the measurement chamber until the operation is in progress, when the difficult implantation of the fluid chamber has been completed. This facilitates the operation and is easy on the sensitive sensor device during the operation.

It is expedient for the interior to be connected via at least one inlet to a probe via which brain fluid can flow from a measuring point of the brain into the interior. In addition, it may be provided that the interior has at least one outlet for brain fluid. The fluid chamber is then part of a shunt system with which brain fluid can be discharged in a manner known per se from the brain, for example, through a drainage pipe into the abdominal cavity.

In a preferred embodiment, it is provided that the fluid chamber comprises a first housing part with a bottom and adjoining side walls and a second housing part which is configured as a cover that is adapted to be placed in a sealed manner on the first housing part.

It is particularly advantageous for one region of the wall of the fluid chamber to be of flexible construction whereas the remaining regions of the wall are of rigid construction. In this way, it is possible to transfer a pressure pulse to the brain fluid in the fluid chamber by pressing in the flexible wall region. This pressure pulse can act as pumping pulse and, therefore, in the event of contamination and clogging of the flow paths, for example, bring about a cleaning. The pressure pulse may, however, also be used, for example, to generate control signals of the sensor and to thereby check whether it is functioning.

In particular, the flexible wall region can be formed by a flexible membrane, for example, a flexible membrane made of silicone.

Such a construction has the further advantage that a cannula for removal of samples of the fluid can be introduced into the interior of the fluid chamber through such a flexible membrane made of silicone or a similar material. In this way, it is possible to gain access to the brain fluid through the scalp and through the flexible membrane even when the measuring device is implanted.

In accordance with a preferred embodiment, the flexible membrane may be inserted into a sealing ring which together with the membrane forms a housing part which is adapted to be fitted together with a second housing part in order to form the fluid chamber. This sealing ring may, for example, be screwed in a sealed manner into the second housing part.

In particular, in a configuration with a flexible wall region of the fluid chamber, it is advantageous for the measurement chamber to be inserted into the fluid chamber in such a way that it is surrounded on all sides by brain fluid. The pumping possibility is thereby improved with the aid of the flexible wall region as the brain fluid also flows through the part of the interior immediately adjacent to the flexible wall region.

The measurement chamber preferably consists of metal, for example, of titanium or a titanium alloy, and it is expedient for the measurement chamber to have rigid walls. Nevertheless, the walls may be relatively thin, for example, the wall thickness lies in the order of magnitude of a few tenths of a millimeter.

The fluid chamber may also consist of a body-compatible metal, for example, of titanium or a titanium alloy. It is, however, also possible to make the fluid chamber from a sterilizable plastic material, for example, from polyetheretherketone. Use of a plastic material has the advantage that the screening effect for the telemetry device in the interior of the measurement chamber, which exists in the case of a metallic construction of the fluid chamber and which impedes transmission of the signals, is eliminated.

The following description of preferred embodiments of the invention serves as a more detailed explanation in conjunction with the drawings.

### BRIEF DESCRIPTION OF THE DRAWINGS

FIG. 1 is a schematic sectional view through the cranium of a patient with an implanted measuring device and an external evaluating device;

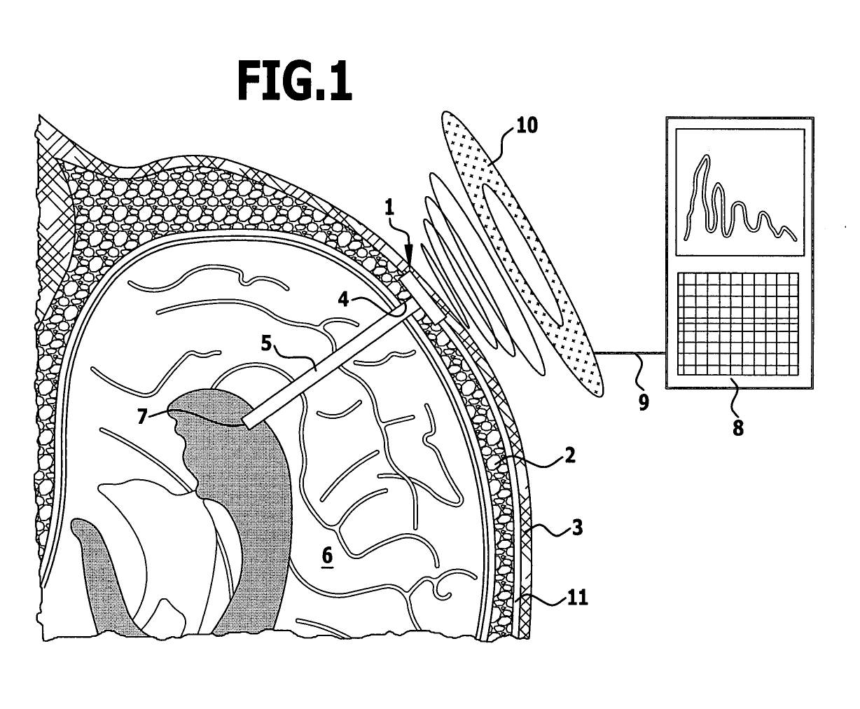

FIG. 2 is a schematic side view of a measuring device with a probe insertable into the brain and with a drainage pipe for the brain fluid in a measuring device insertable over a drill hole in the cranial vault;

FIG. 3 is a view similar to FIG. 2 in a measuring device implantable next to a drill hole in the cranial vault;

FIG. 4 is a sectional view of a closed fluid chamber without the measurement chamber inserted;

FIG. 5 is a view similar to FIG. 4 with the measurement chamber inserted;

FIG. 6 is a longitudinal sectional view through a measurement chamber;

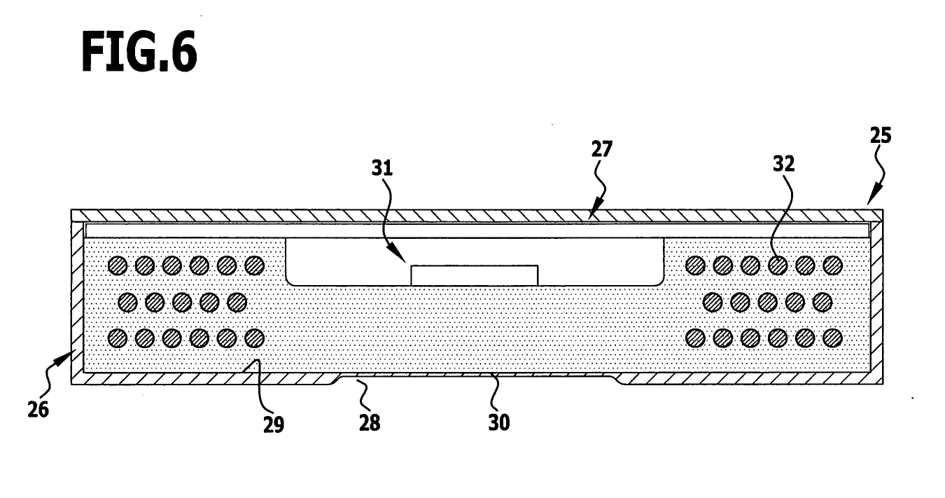

FIG. 7 is a sectional view of an empty and unclosed measurement chamber;

FIG. 8 is a perspective view of a closed measurement chamber;

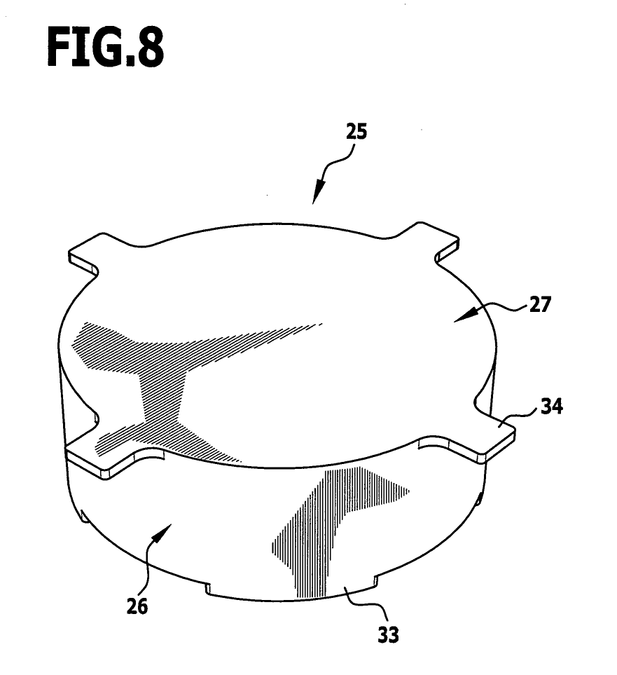

FIG. 9 is a longitudinal sectional view similar to FIG. 5 of a measuring device through which brain fluid is flowing;

FIG. 10 is a view similar to FIG. 9 representing the flow of the brain fluid when a flexible wall region is pressed in; and

FIG. 11 is an exploded view of a further preferred embodiment of a measuring device.

### DETAILED DESCRIPTION OF THE INVENTION

The measuring device 1 shown in the drawings is usually implanted outside the cranial bone 2 and beneath the scalp 3 over a drill hole 4 or next to such a drill hole. A probe 5 or a catheter passes through the drill hole 4 into the interior of the brain 6 and terminates at an inlet point 7, at which brain fluid from the brain 6 can enter the probe 5. This brain fluid is fed by the probe 5 to the measuring device 1, so that physiological values of the brain fluid, for example, the pressure of the brain fluid and/or the temperature can be measured in the measuring device 1.

The measured values of the physiological parameters recorded in this way in the measuring device 1 are transmitted in a wireless manner to an external evaluating device which is connected by a measurement line 9 to a coil 10. This coil 10 can be so arranged in the proximity of the measuring device 1 that it can exchange signals with a corresponding coil of the measuring device in a wireless manner. The measuring device 1 can also be supplied with external power via these coils.

Brain fluid fed to the measuring device 1 can be discharged through a drainage pipe 11. Such a drainage pipe terminates, for example, in the abdominal cavity of the patient. The probe 5, the measuring device 1 and the drainage pipe 11 therefore form a shunt system with which brain fluid can be removed from the cranium.

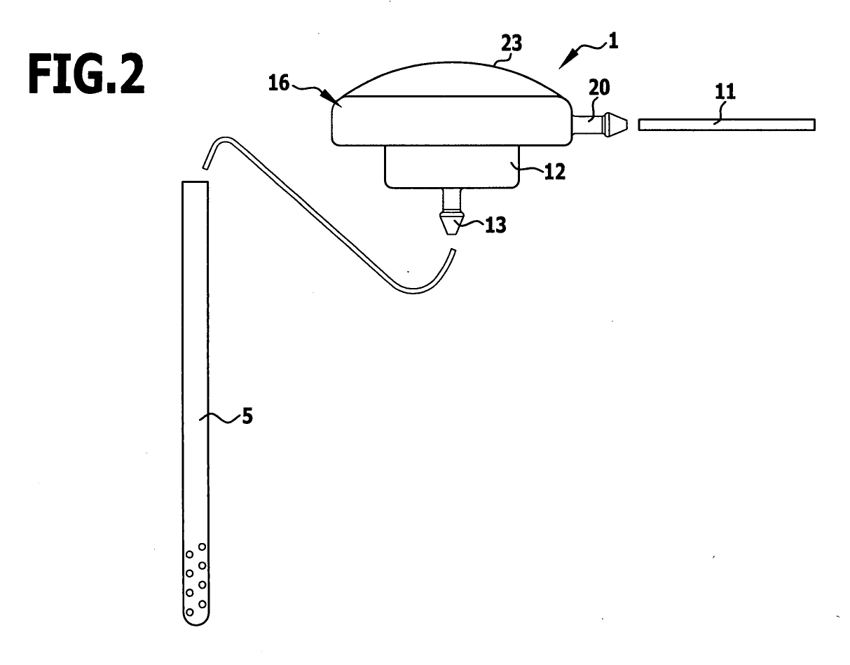

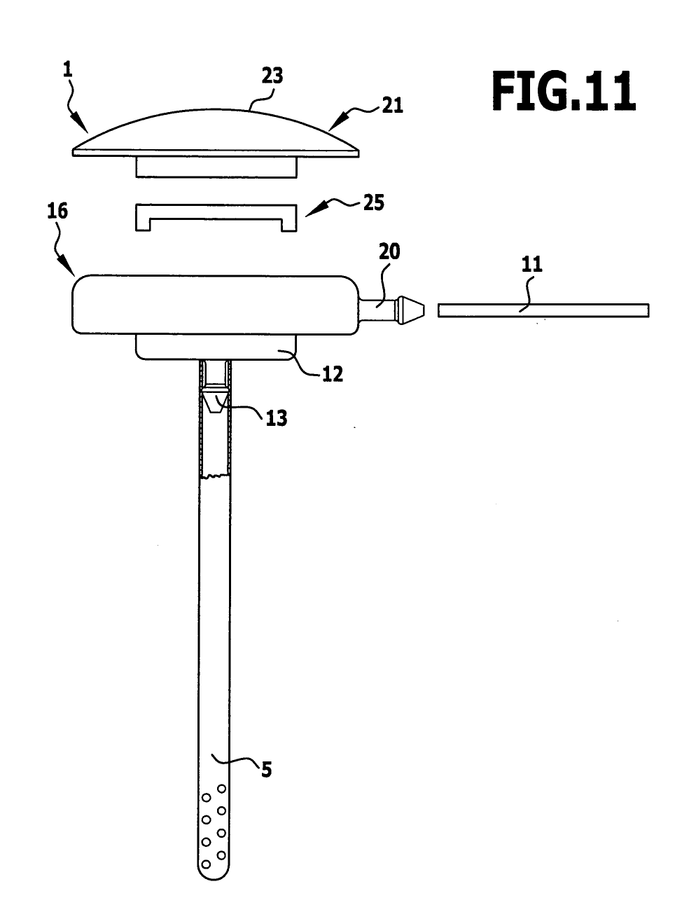

If the measuring device 1 is placed directly over the drill hole 4, it is then expedient for the measuring device 1 to have on its underside a projection 12 entering the drill hole 4 and an adjoining connection 13 which, for example, can be connected by a flexible line 14 to the probe 5 (FIG. 2) or onto which the probe 5 can be directly pushed (FIG. 11).

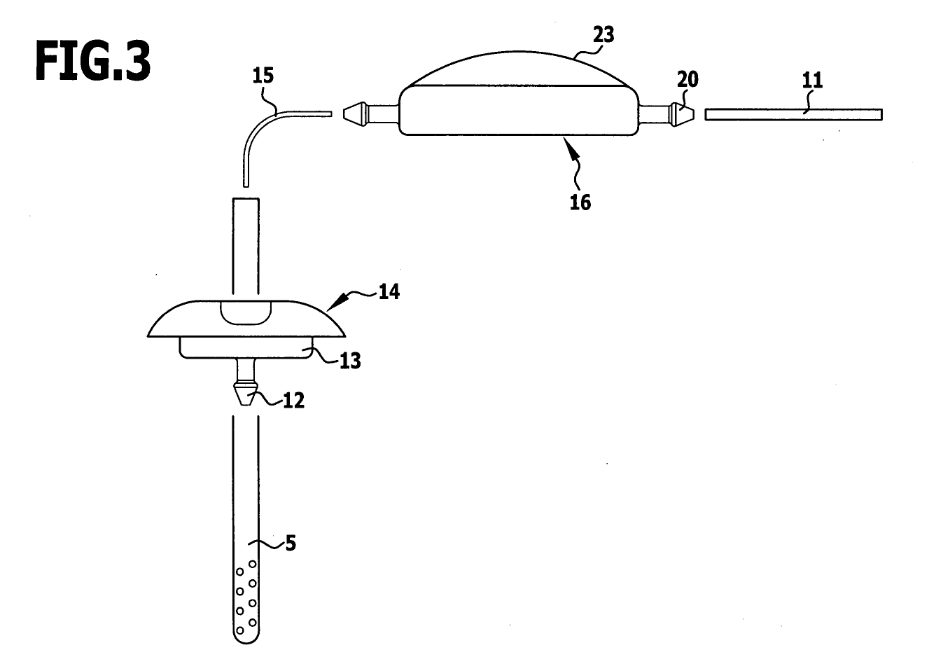

If, in contrast, the measuring device 1 is arranged next to a drill hole 4, it is advantageous for a drill hole cover 14 to be provided, which also carries a projection 12 and a connection 13 on its underside, and which diverts the brain fluid parallel to the cranial bone, so that the brain fluid enters the measuring device 1 at the side through a further line 15 (FIG. 3).

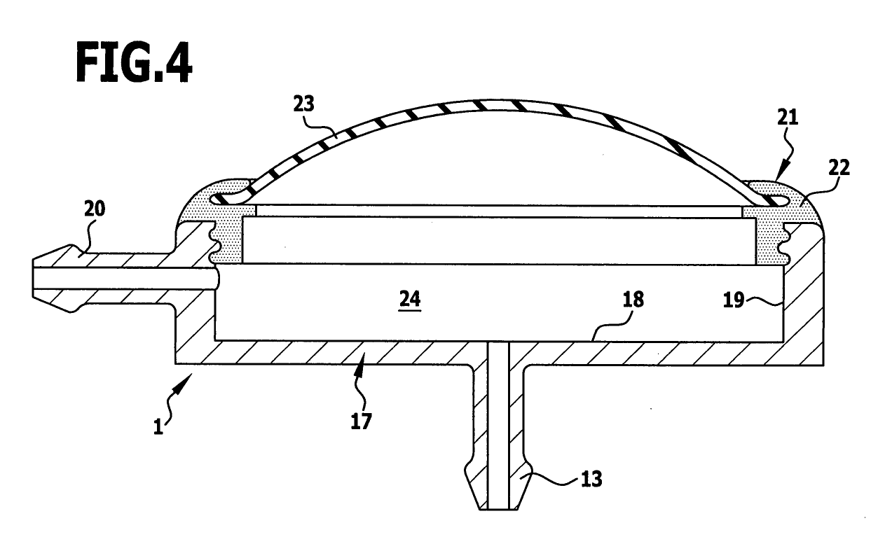

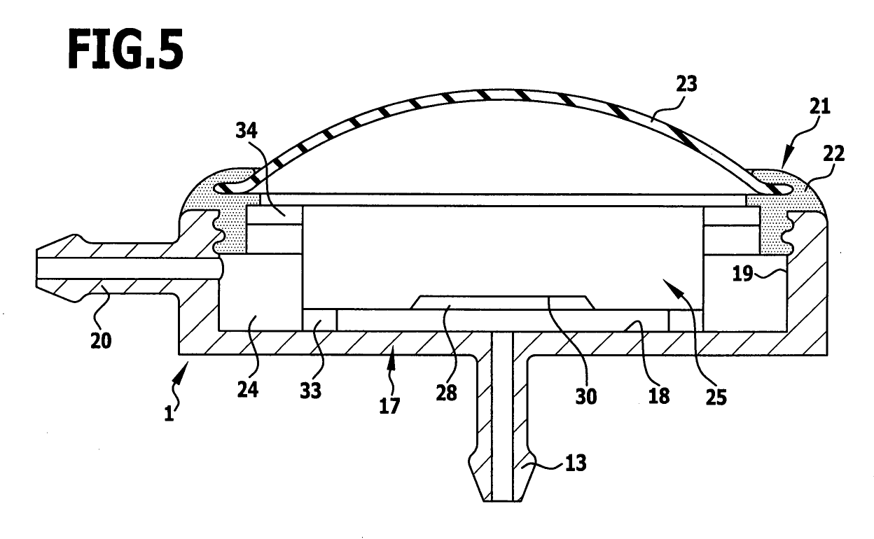

The measuring device 1 comprises a fluid chamber 16 with a can-shaped base part 17 with a flat bottom 18 and adjoining perpendicular walls 19 on the outside. This can-shaped base part 17 preferably has a circular cross section. In the embodiment shown in FIGS. 4 and 5, the bottom 18 has a downwardly directed connection 13 for admission of the brain fluid and an outlet 20 extending horizontally out of the wall 19 for discharge of the brain fluid.

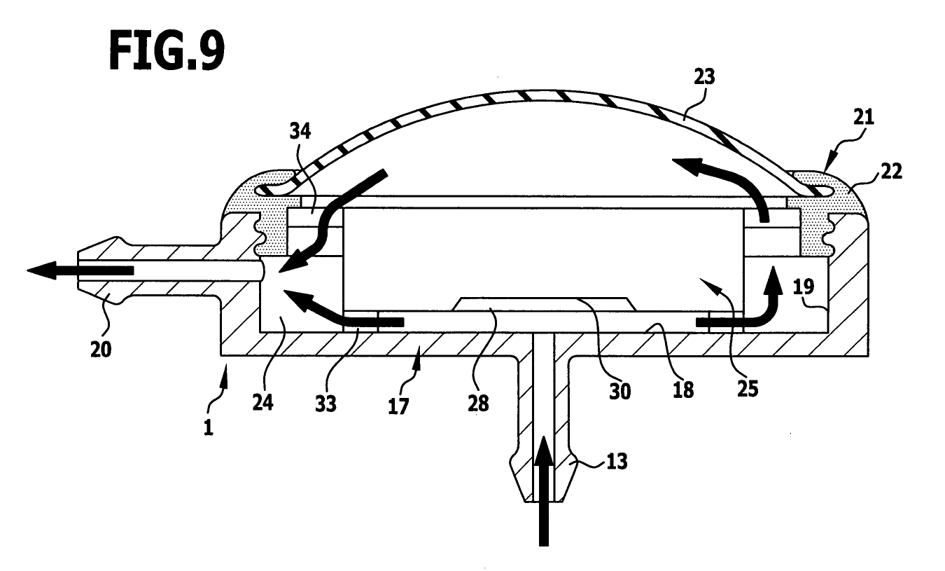

The can-shaped base part 17 is open on its upper side where it can be closed tight by a lid-shaped housing part 21. This lid-shaped housing part 21 comprises a ring 22, which is configured as a sealing ring and is sealingly connectable to the wall 19 of the base part 17, for example, by being screwed in or glued in. Inserted in a sealed manner into the ring 22 is a hood-shaped flexible membrane 23, which preferably consists of silicone, whereas the ring 22 may, for example, consist of polyetheretherketone. When fitted together, the bottom-shaped base part 17 and the lid-shaped housing part 21 form a housing-like fluid chamber with an interior 24, which is closed except for the connection 13 and the outlet 20. In the implanted state, the brain fluid flows through this interior 24 and fills it out completely. The flexible membrane 23 is thereby arched outwards in the manner shown in FIG. 9.

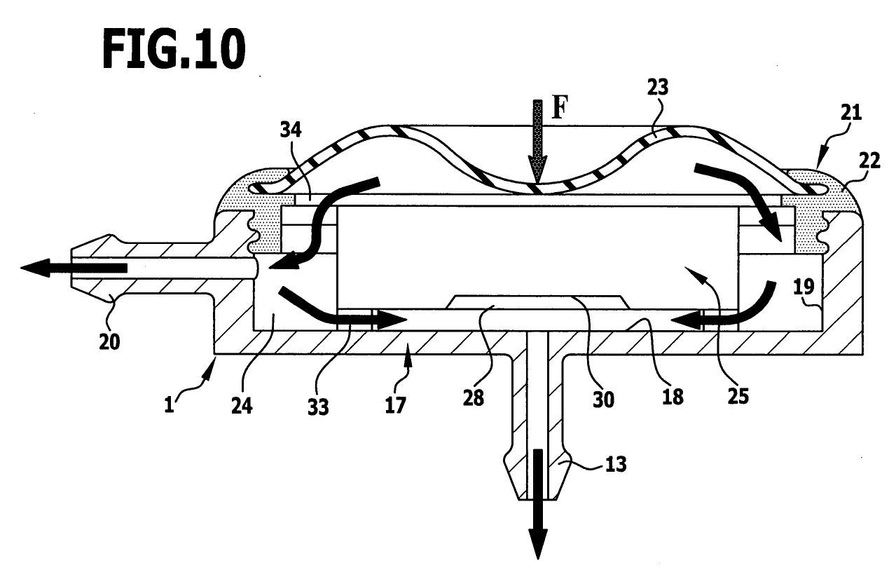

This flexible membrane can be pushed inwards, as shown in FIG. 10, by pressure on the membrane 23. The brain fluid is thereby expelled from the interior 24 through both the outlet 20 and the connection 13 out of the fluid chamber 16, i.e., a backflushing of the probe 5 takes place and an increased flushing of the drainage pipe 11. This flushing effect may be used to clean the corresponding pipes, however, it is also possible to transmit by means of this pressure on the flexible membrane 23 a pressure pulse onto the brain fluid, which can then be registered by the measuring device 1. It is thereby possible to check whether the measuring device is functioning.

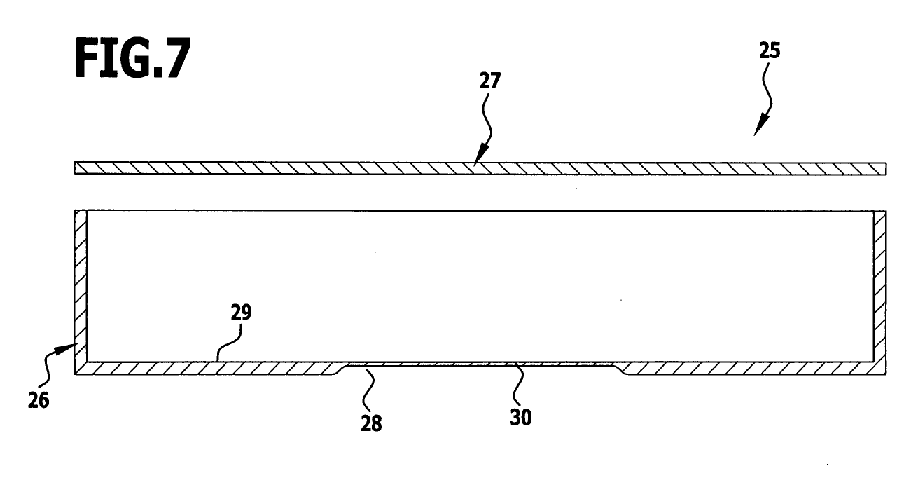

Inserted in the interior 24 is a completely closed-off measurement chamber 25 comprising a base part 26 which is likewise can-shaped and has a circular cross section, and a cover 27 which is placed in a sealed manner on this base part 26 (FIG. 7). This measurement chamber preferably consists of metal, in particular, of titanium or a titanium alloy. The walls are of rigid construction except for a central region 28 on the bottom 29 of the base part 26. In this region, either a flexible membrane 30 is inserted or the bottom 29 is of such thin construction owing to the removal of material that it forms a flexible membrane 30.

Accommodated in the interior of the measurement chamber 25 are a sensor chip 31 and a coil 32 surrounding this. The sensor chip 31 carries at least one sensor, for example, a pressure sensor and/or a temperature sensor, in addition, electronic components for processing and digitizing the electric signals generated by the sensors and for supplying power, and also electronic components of a telemetry circuit which transmits the signals generated by the sensors via the coil 32 to the outside. These signals can then be received by the coil 10 of the external evaluating device 8.

The interior of the measurement chamber 25 is filled with a pressure-transferring medium, for example, with air, with an oil or with a gel. It is also possible for mechanical transfer members, for example, pistons or levers to be provided between the flexible membrane 30 and the sensors on the sensor chip 31. It is only essential that the pressure of the brain fluid be able to reach the sensors on the sensor chip 31 via the flexible membrane 30 and result in corresponding measurement signals there.

The measurement chamber 25 carries at its lower end supporting projections 33 extending over part of the circumference and projecting downwardly, and on its cover 27 centering webs 34 which protrude radially so that the measurement chamber 25 is arranged in a precisely defined position when inserted into the base part 17 of the fluid chamber 16. The supporting projections 33 are then supported on the bottom 18 of the base part 17, and the radial centering webs 34 either on the wall 19 of the base part 17 or after closure of the fluid chamber on the ring 22. At any rate, it is ensured by this arrangement that the brain fluid can flow all around the measurement chamber 25, i.e., the measurement chamber 25 is spaced all over from the bottom 18, from the walls 19 and also from the lid-shaped housing part 21, so that the brain fluid can flow unimpeded through these spaces.

This is particularly important when a backflushing pulse or a pressure pulse is to be transmitted to the brain fluid by means of the flexible membrane 23, but it is also of importance that no distortions of the pressure measurement occur as a result of the brain fluid being able to flow freely through the interior 24.

The measurement chamber 25 can be inserted into the fluid chamber 16 after the latter has been implanted. It is also readily possible to exchange the measurement chamber, should malfunctions of the measurement chamber occur or should other measurements be of interest.

1. Measuring device for physiological parameters, comprising an implantable fluid chamber accommodating brain fluid, a sensor device communicating with the brain fluid in the fluid chamber via a membrane and having a sensor for one or more physiological parameters, electronic components and a telemetry device for wireless transmission of signals of the sensor, wherein the fluid chamber comprises at least two housing parts which are adapted to be fitted together in a sealed manner, thereby forming a closed interior, and which allow access to the interior when not fitted together, and wherein the sensor device is arranged in a measurement chamber closed on all sides, which is configured as a component that is adapted for independent handling and is insertable in a defined position in the interior of the fluid chamber.

2. Measuring device in accordance with claim 1, wherein the interior is connected via at least one inlet to a probe via which brain fluid can flow from a measuring point of the brain into the interior.

3. Measuring device in accordance with claim 1, wherein the interior has at least one outlet for brain fluid.

4. Measuring device in accordance with claim 1, wherein the fluid chamber comprises a first housing part with a bottom and adjoining side walls and a second housing part which is configured as a cover that is adapted to be placed in a sealed manner on the first housing part.

5. Measuring device in accordance with claim 1, wherein one region of the wall of the fluid chamber is of flexible construction whereas the remaining regions of the wall are of rigid construction.

6. Measuring device in accordance with claim 5, wherein the flexible wall region is formed by a flexible membrane.

7. Measuring device in accordance with claim 6, wherein the flexible membrane consists of silicone.

8. Measuring device in accordance with claim 5, wherein the flexible membrane is inserted into a sealing ring which together with the membrane forms a housing part which is adapted to be fitted together with a second housing part in order to form the fluid chamber.

9. Measuring device in accordance with claim 1, wherein the measurement chamber is inserted into the fluid chamber in such a way that it is surrounded on all sides by brain fluid.

10. Measuring device in accordance with claim 9, wherein fixing projections which are part of the fluid chamber and/or the measurement chamber are arranged between the fluid chamber and the measurement chamber for securing the measurement chamber in the fluid chamber.

11. Measuring device in accordance with claim 1, wherein the measurement chamber consists of metal.

12. Measuring device in accordance with claim 1, wherein the measurement chamber has rigid walls.

13. Measuring device in accordance with claim 1, wherein the fluid chamber consists of a sterilizable plastic material.

14. Measuring device in accordance with claim 13, wherein the fluid chamber consists of polyetheretherketone.
